(19) United States
(12) Patent Application Publication
(10) Pub. No.: US 2015/0337010 A1
(43) Pub. Date: Nov. 26, 2015

(54) Method for treating or preventing liver related diseases

(71) Applicant: Tunghai University, Taichung (TW)

(72) Inventors: Wen-Dee CHIANG, Taichung (TW); Wan-Teng LIN, Taichung (TW)

(21) Appl. No.: 14/327,070

(22) Filed: Jul. 9, 2014

(30) Foreign Application Priority Data
May 23, 2014 (TW) 103118129

(51) Int. Cl. C07K 5/10; A23L 1/305

(52) U.S. Cl. CPC C07K 5/10; A23L 1/3053; A23V 2002/00

(57) ABSTRACT

This invention discloses an isolated peptide containing an amino acid sequence of SEQ ID No.1. The isolated peptide is capable of providing the effects in suppressing hepatocyte apoptosis, attenuating hepatic triglyceride accumulation, suppressing the expression of inflammatory cytokines, inhibiting expression of pro-apoptotic proteins, enhancing expression of survival factor and inhibiting the expression of biomarker of hepatic fibrosis. Therefore, this invention discloses the peptide as the effective ingredient in a composition for treating or preventing liver related diseases.

The current application claims a foreign priority to the patent application of Taiwan No. 103118129 filed on May 23, 2014.

### FIELD OF THE INVENTION

This invention relates to a use of isolated peptide, especially relates to a method for treating or preventing liver related diseases.

### DESCRIPTION OF THE RELATED ART

According to the previous reports, Non-alcoholic fatty liver disease (NAFLD) resulted from massive hepatic triglyceride accumulation is one of the most common metabolic syndromes. NAFLD would further progresses toward nonalcoholic steatohepatitis, hepatic fibrosis, cirrhosis and hepatic carcinomas (HCC).

Because liver is not the organ participating in fat storage, the concentration of hepatic triglyceride is maintained at the minimal level in normal physiological condition. However, there is a considerable movement of triglyceride and fatty acids into and out of liver in response to feeding and fasting. Therefore, both hypernutrition or insulin resistance would cause the imbalanced lipid uptake and usage of hepatocytes. The imbalanced lipid uptake and usage in liver will result in the excessive hepatic triglyceride accumulation.

Moreover, the lipid oxidation is preferentially carried out upon β-oxidation response occurring in peroxisome and mitochondria. Therefore, the damages occurred in peroxisome and mitochondria would disturb the progression of β-oxidation reaction. According to previous studies, the expression of peroxisome proliferators-activated receptors (hereafter referred to as PPARs) shows correlation with fatty acids oxidation. Herein, peroxisome proliferators-activated receptor α (hereafter referred to as PPARα) plays the critical role for adjusting fatty acid oxidation and regulating energy metabolism. In addition, PPARα regulates the expression of genes encoding the majority of enzymes for β-oxidation pathways in peroxisome and mitochondria. Therefore, activation of PPARα would elevate fatty acid metabolism rate to control the hepatic triglyceride accumulation through reducing lipid accumulation in serum and hepatocytes.

The previous studies show that peroxisome proliferators-activated receptor γ (hereafter referred to as PPARγ) plays a critical role in promoting hepatocyte differentiation. It is also capable of improving recovery of the liver damage resulted from NAFLD through restoring the insulin sensitivity of hepatocytes. In addition, PPARγ not only activates expression of the proteins involving in fatty acid uptake, fatty acid transport and fatty acid synthesis. The expression of PPARγ is also capable of avoiding the inflammation of hepatocytes through suppressing the expression of pre-inflammatory cytokines such as TNFα and IL-6. In addition, PPARγ is also required for activating glycogenesis due to the function in activation of PGC-1 expression by cooperation with Forkhead box O-1 (hereafter referred to as FOX-O1).

In addition to lipid accumulation, hepatocyte apoptosis is another characteristic of NAFLD. The hepatocyte apoptosis would further induce the disorders such as hepatic injury, severe hepatic inflammation and cirrhosis. Therefore, the secretion of inflammatory cytokines from the hepatocytes represents to hepatic injury that would affect the progression of NAFLD. Two pathways including intrinsic pathway and extrinsic pathway majorly induce the apoptosis. The intrinsic pathway involves the disruption of mitochondria membrane potential and release of cytochrome c that triggers caspase-9. The extrinsic pathway of apoptosis involves the activation of death receptors on cell membrane such as Fas receptor, recruitment of the adaptor molecule Fas-associated death domain (hereafter referred to as FADD) and activation of Caspase-8. In addition, the members of Bcl-2 family on the mitochondrial membrane also involve in the regulation of apoptosis, herein, B-cell lymphoma-2 protein (hereafter referred to as Bcl-2) is an anti-apoptotic factor and Bcl-2 associated X protein (hereafter referred to as Bax) is a pro-apoptotic factor.

Collectively, it is considerable to propose that protein expression levels of PPARα, PPARγ, PGC-1 and Bcl-2 are decreased in the patients bearing fatty liver or hepatic injury. In contrast, the protein expressions of tumor necrosis factor α (hereafter referred to as TNFα), interleukin-6 (hereafter referred to as IL-6), cytochrome c, Caspase-3, Caspase-8, Caspase-9 and Bax are increased in those indicated patients. Furthermore, insulin-like growth factor I receptor (hereafter referred to as IGFIR) protein regulates the secretion of inflammatory cytokines and promotes the activation of immunocyte by activation of PISK/AKT pathway through an indirect manner. Therefore, the increased protein level of cell survival factors including IGFIR, phosphatidylinositol 3-kinase (hereafter referred to as PI3K) and serine threonine kinase (hereafter referred to as AKT) are indicators to adjust how the hepatocytes recovered from hepatic injury caused by fatty liver or obesity.

Although the apoptosis of hepatocyte reveals important correlation with hepatic disorders including fatty liver, hepatic cirrhosis and hepatic fibrosis, the appropriate drug for management of NAFLD is still lacked. In other words, the effective method to prevent fatty liver and the related hepatic disorders in clinic is required. Therefore, it is a critical issue to develop the efficient method for therapy or prevention of fatty liver and the related hepatic diseases due to the high incidence and high risk of fatty liver.

### SUMMARY OF THE INVENTION

The present invention is to provide the method for treating or preventing liver related diseases, which by administration to a subject an composition including an effective ingredient, an isolated peptide containing an amino acid sequence of SEQ ID No.1. According to the purpose of the invention, the method is capable of curing or preventing the hepatic diseases or the related disorders, especially fatty liver, hepatic inflammation, hepatic fibrosis, hepatic injury, or hepatocytes apoptosis occurred by the above mentioned diseases.

Another purpose of the invention, administrating the peptide to the subject can avoid the side effects and improve the absorptivity in the subject.

In a still further purpose of the invention, the isolated peptide can be prepared by simply biotechnological method or chemical method, so the isolated peptide can be provided in lower production cost and having more stable quality and efficacy.

An embodiment of the present invention is to provide the method for treating or preventing liver related diseases, which comprises administering to a subject a composition including an effective amount of an isolated peptide containing an amino acid sequence of SEQ ID No.1 and at least one medical, healthy or food acceptable vehicle.

In an embodiment of this present invention, the liver related diseases can be hepatic fibrosis, fatty liver, hepatic inflammation, hepatic injury or apoptosis of hepatocytes.

In an embodiment of this present invention, the amino acid sequence of the isolated peptide is SEQ ID No.1.

### BRIEF DESCRIPTION OF THE DRAWINGS

FIG. 1 shows the change of body weight in the mice of the group 1 and the group 2. The results represent mean±SD of three independent experiments.

FIG. 2A shows the LDL-cholesterol levels in the mice of the each group. The results represent mean±SD of three independent experiments.

FIG. 2B shows the triglyceride levels in the mice of the each group. The results represent mean±SD of three independent experiments.

FIG. 3A shows the liver tissue architecture from the mice of the group 1 by H&E staining of the hepatic histology.

FIG. 3B shows the liver tissue architecture from the mice of the group 2 by H&E staining of the hepatic histology.

FIG. 3C shows the liver tissue architecture from the mice of the group 3 by H&E staining of the hepatic histology.

FIG. 3D shows the liver tissue architecture from the mice of the group 4 by H&E staining of the hepatic histology.

FIG. 3E shows the liver tissue architecture from the mice of the group 5 by H&E staining of the hepatic histology.

FIG. 4A shows the microvesicular and macrovesicular steatosis of the mice liver in the group 1 by H&E staining.

FIG. 4B shows the microvesicular and macrovesicular steatosis of the mice in the group 2 by H&E staining.

FIG. 4C shows the microvesicular and macrovesicular steatosis of the mice in the group 3 by H&E staining.

FIG. 4D shows the microvesicular and macrovesicular steatosis of the mice in the group 4 by H&E staining.

FIG. 4E shows the microvesicular and macrovesicular steatosis of the mice in the group 5 by H&E staining.

FIG. 5 shows the protein expressions of TNFα and IL-6 in mouse liver of the each group.

FIG. 6A shows the quantified expression levels of TNFα in mouse liver of the each group. The quantified expression level of TNFα is normalized with tubulin, first. The results represent mean±SD of three independent experiments.

FIG. 6B shows the quantified expression levels of IL-6 in mouse liver of the each group. The quantified expression level of IL-6 is normalized with tubulin, first. The results represent mean±SD of three independent experiments.

FIG. 7 shows the protein expressions of Bcl-2, Bax, Cap-9, and cytochrome c in mouse liver of the each group.

FIG. 8A shows the quantified expression levels of Bcl-2 in mouse liver of the each group. The quantified expression level of Bcl-2 is normalized with tubulin, first. The results represent mean±SD of three independent experiments.

FIG. 8B shows the quantified expression levels of Bax in mouse liver of the each group. The quantified expression level of Bax is normalized with tubulin, first. The results represent mean±SD of three independent experiments.

FIG. 8C shows the quantified expression levels of cytochrome c in mouse liver of the each group. The quantified expression level of cytochrome c is normalized with tubulin, first. The results represent mean±SD of three independent experiments.

FIG. 8D shows the quantified expression levels of caspase-9 in mouse liver of the each group. The quantified expression level of caspase-9 is normalized with tubulin, first. The results represent mean±SD of three independent experiments.

FIG. 9 shows the protein expressions of caspase-3 and caspase-8 in mouse liver of the each group. The expression of tubulin is utilized as the internal control among different samples.

FIG. 10A shows the quantified expression levels of caspase-8 in mouse liver of the each group. The quantified expression level of caspase-8 is normalized with tubulin, first. The results represent mean±SD of three independent experiments.

FIG. 10B shows the quantified expression levels of Caspase-3 in mouse liver of the each group. The quantified expression level of Caspase-3 is normalized with tubulin, first. The results represent mean±SD of three independent experiments.

FIG. 11 shows the protein expressions of survive related factors including pIGF-IR, IGF-IR, pPI3K, PI3K, pAKT and AKT in mouse liver of the each group. The expression of tubulin is utilized as the internal control among different samples.

FIG. 12A shows the quantified expression levels of pIGF-IR in mouse liver of the each group. The quantified expression level of pIGF-IR is normalized with tubulin, first. The results represent mean±SD of three independent experiments.

FIG. 12B shows the quantified expression levels of PI3K in mouse liver of the each group. The quantified expression level of PI3K is normalized with tubulin, first. The results represent mean±SD of three independent experiments.

FIG. 12C shows the quantified expression levels of pAKT in mouse liver of the each group. The quantified expression level of pAKT is normalized with tubulin, first. The results represent mean±SD of three independent experiments.

FIG. 13 shows the protein expression of MMP-9 in mouse liver of the each group. The expression of tubulin is utilized as the internal control among different samples.

FIG. 14 shows the quantified expression level of MMP-9 in mouse liver of the each group. The quantified expression level of MMP-9 is normalized with tubulin, first. The results represent mean±SD of three independent experiments.

FIG. 15 shows the protein expression of PPARα in mouse liver of the each group. The expression of tubulin is utilized as the internal control among different samples.

FIG. 16 shows the quantified expression levels of PPARα in mouse liver of the each group. The quantified expression level of PPARα is normalized with tubulin, first. The results represent mean±SD of three independent experiments.

### DETAILED DESCRIPTION OF THE INVENTION

This present invention is to provide an isolated peptide containing the amino acid sequence as shown in SEQ ID no. 1. The isolated peptide is capable to inhibit hepatocytes apoptosis, reduce massive triglyceride accumulation, inhibit the protein expression of pro-inflammatory factors, activate the protein expression of survival factors, and inhibit the expressions of the biomarkers for hepatic fibrosis.

Moreover, the peptide can be isolated from an organism by extraction, purification or hydrolyzation, or obtained by methods of chemical synthesis. A skilled person in the art related to this present invention should understand that it is allowed to modify the peptide for improving the stability or the functions without breaking the peptide in normal physiological condition, for example, the additional peptide is used to modify N′-end or C′-end of the peptide disclosed in the present invention.

The term “composition” includes, but not limits to, pharmaceutical composition, nutritional addictive composition or healthy composition. Furthermore, the composition can be in different forms depending on the way of composition delivery, for example, the forms include, but not limit in, drops, powder, injection, pill, pistil, patches, orally liquid and so on.

The term “effective amount” means the amount (weight percentage of composition) of the bioactive ingredient (extract or compound) for generating specific effect, preventing and/or treating effect. The person skilled in the art related to this present invention should understand that the effective amount can be different because of reasons such as trying to reach specific effect, to prevent and/or treat the kind of diseases and the way to deliver compositions. For generally, the amount of the bioactive ingredient in compound can be about 1% to about 100% of the weight of the composition, better is about 30% to 100%.

The term “medical, healthy or food acceptable vehicle” includes any standard medical, healthy or food acceptable vehicle. The vehicle that can be solid or liquid depends on the form of pharmaceutical, nutritional addictive or healthy composition. Examples of the solid vehicle include lactose, sucrose, gelatin and agar. Examples of the liquid vehicle include normal saline, buffered saline, water, glycerol and methanol.

Embodiments of this present invention are further described with the following examples, but not limited to it. The purposes, features and advantages of this present invention will become more clarify because of the following description and figures.

### Example 1

### Animal Experiments

The experiments using the experimental mice were conducted according to the IACUC-100-12 protocol approved by Academia Sinica Institutional Animal Care and Utilization Committee (IACUC) ethics committee. All C57BL/6 male mice (6-weeks-old) were randomly divided into 5 groups that contain 8 mice in each. These mice were individually housed in a room temperature at 24±2° C. and 55±10% humidity with 12 hours of light cycle. Herein, the control mice in the group 1 were fed with standard laboratory diet and administrated 0.9% normal saline by intra-peritoneal injection. In the group 2, the mice were fed with high-fat diet to induce the fatty liver, and administrated 0.9% normal saline by intra-peritoneal injection. In the group 3, the mice were fed with high-fat diet and administrated with peptide of SEQ ID No.1 by intra-peritoneal injected with the dose of 5 mg/kg/day. In the group 4, the mice were fed with high-fat diet and administrated with peptide of SEQ ID No.1 by intra-peritoneal injected with the dose of 15 mg/kg/day. In the group 5, the mice were fed with high-fat diet and administrated with peptide of SEQ ID No.1 by intra-peritoneal injected with the dose of 25 mg/kg/day. The inter-peritoneal injections were performed on these mice between third to sixth weeks of culture.

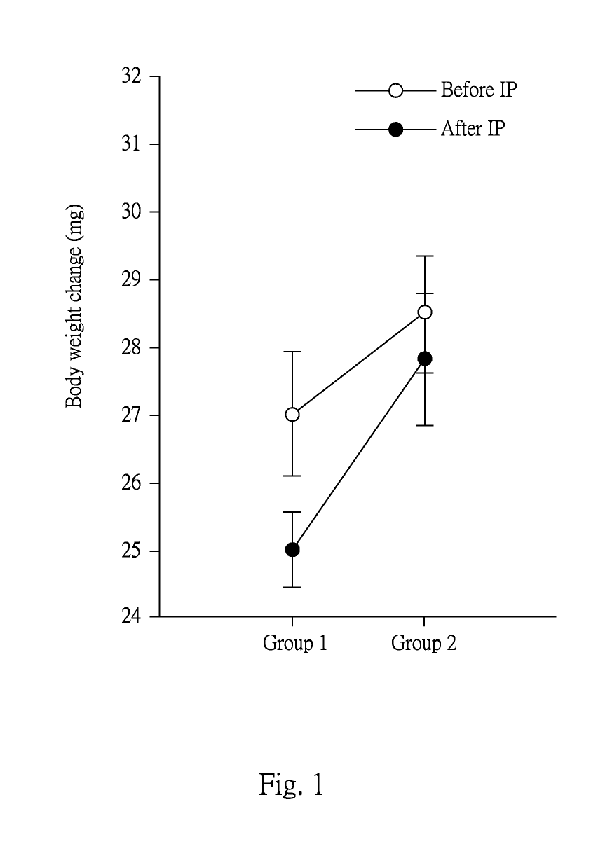

The altered body weight of the mice in the group 1 and the group 2 were recorded for statistic calculation as shown in FIG. 1. The statistic result in FIG. 1 showed the obvious increase in the body weight of mice in the group 2 with comparison of that in the group 1. The increased body weight of mice in the group 2 suggests that feeding with high-fat diet cause obesity in mice. After the conditional feeding, the mice subjected for blood sampling were sacrificed for the following examples of this invention.

### Example 2

### Analysis of the Composition of Fat in Mice

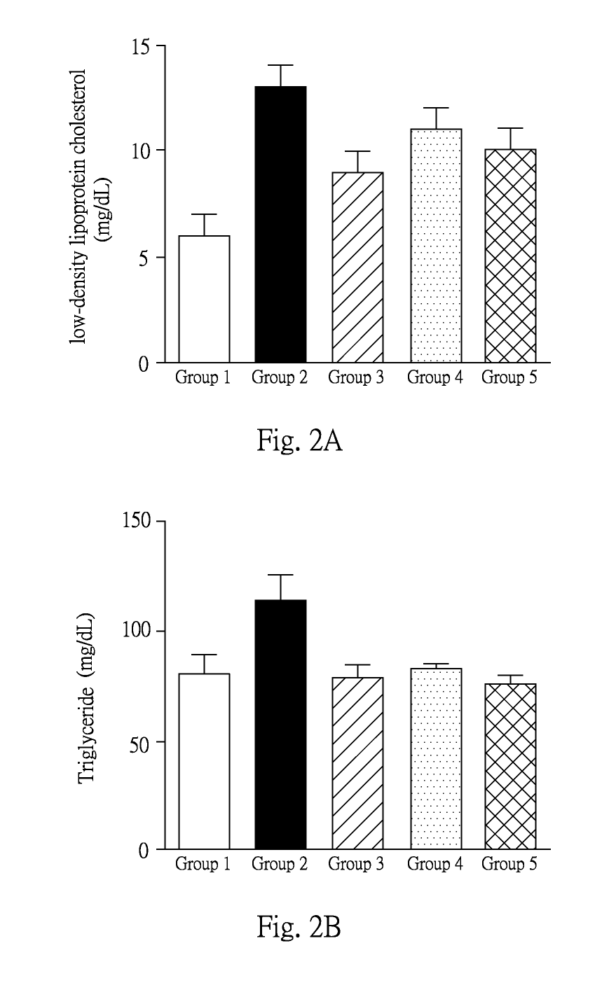

The blood samples collected from the mice in each group indicated in example 1 into the microcentrifuge tubes containing heparin (10 μL, 1000 IU/ml). The blood plasma was separated by centrifugation at 10000 rpm for 10 minutes. The concentration of low density lipoprotein-cholesterol (hereafter referred to as LDL) and triglyceride in the isolated blood plasma from the mice in the each group were determined by using the commercial detection kit. The measured concentrations of LDL and triglyceride in the blood plasma were shown in FIG. 2A and FIG. 2B. The results in FIG. 2 revealed that LDL and triglyceride of the mice in the group 2 were obviously increased with comparison of that in the group 1. Comparing with the group 2, the administration of the peptide of SEQ ID No.1 to the mice in any one of the groups 3˜5 revealed the obviously decreased concentration of LDL and triglyceride. According to the above results, it indicates that the treatment of the peptide disclosed in this present invention is able to efficiently reduce the concentration of LDL and triglyceride in blood plasma.

### Example 3

### Histopathological Examination of Mouse Liver

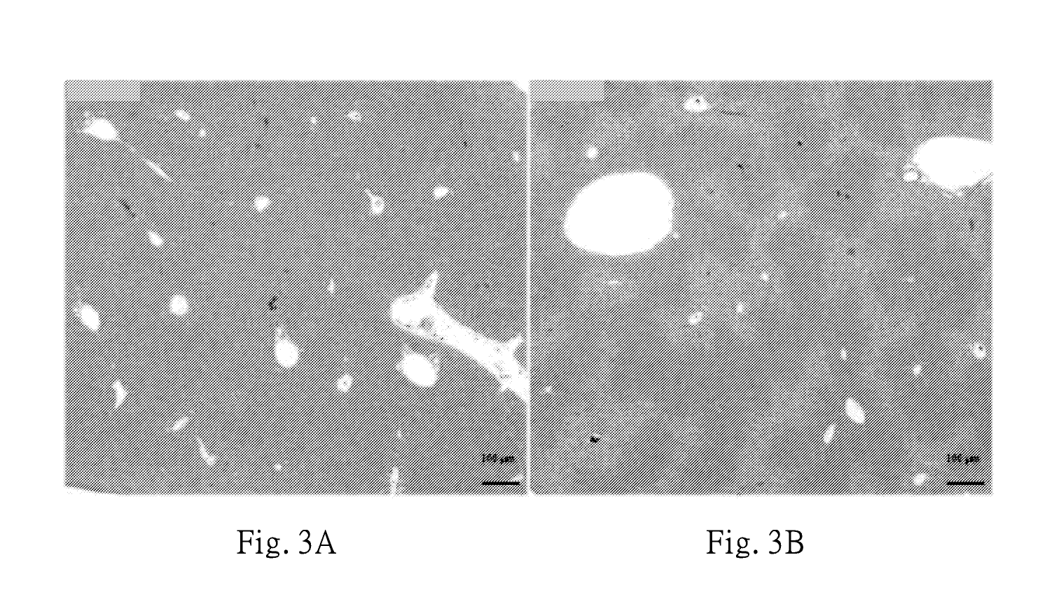

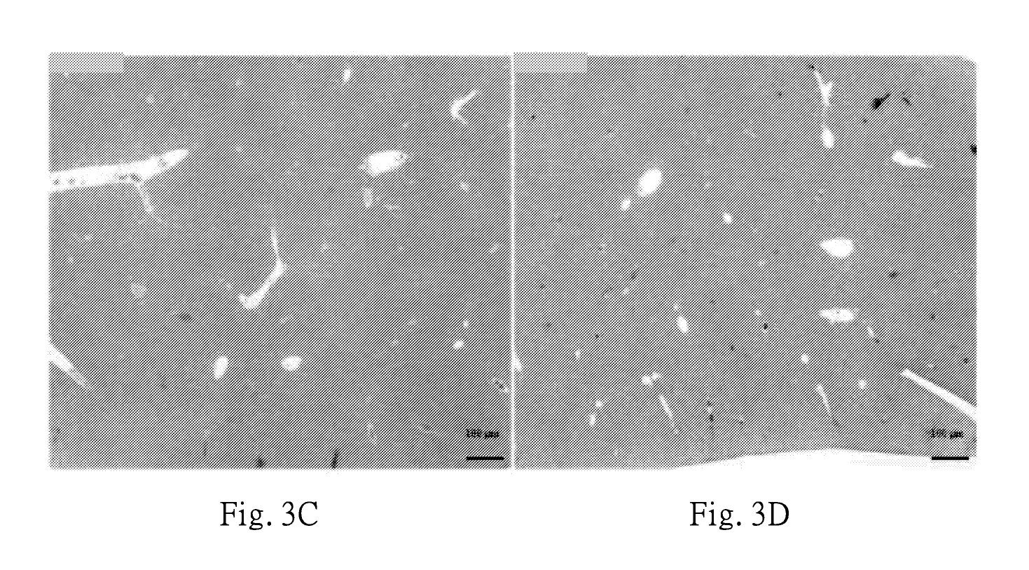

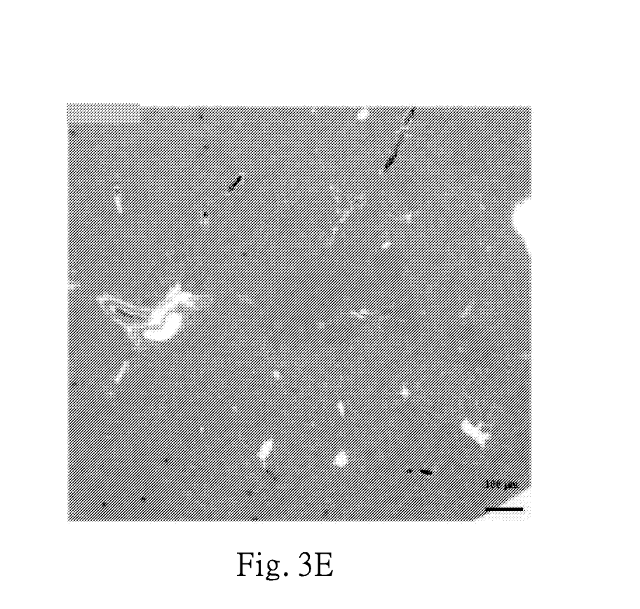

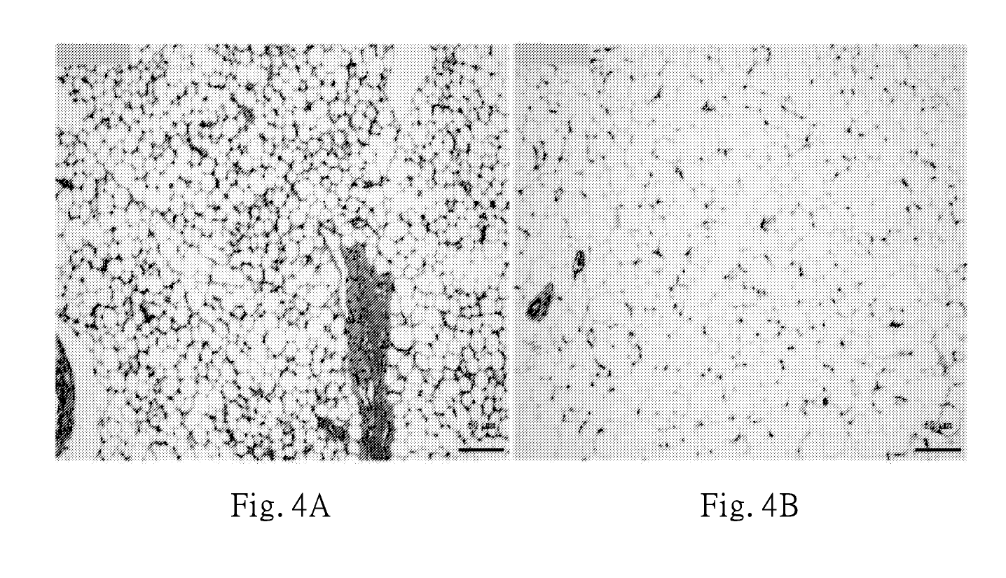

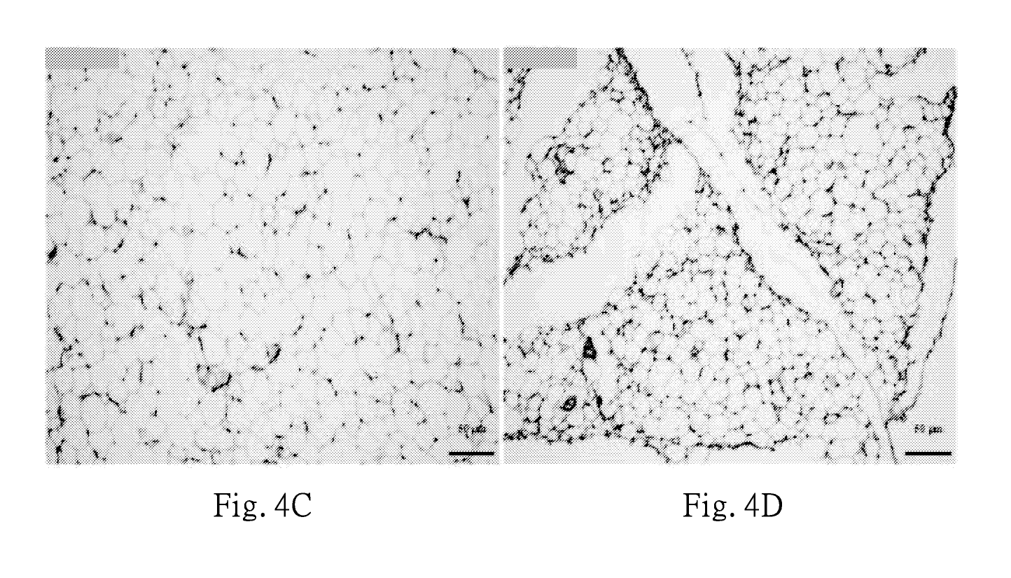

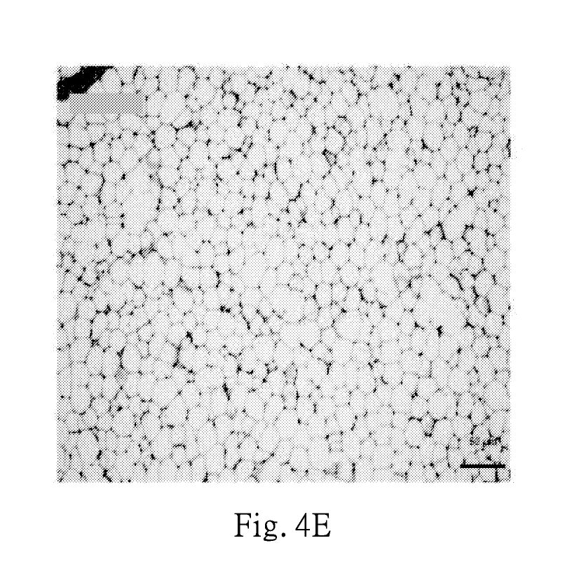

The livers were excised from the mice of the each group; soaked in formalin; dehydrated by passing consecutively through 100%, 95% and 75% alcohol and then embedded in paraffin wax. The each embedded liver tissue block was cut into 0.2 μm-thick sections and deparaffinization by soaking in xylene. The histological section of the mouse liver in the each group was consecutively stained by hematoxylin and eosin (H&E) and rinsed with water. The photomicrographs of the histological section of the mouse liver in the each group obtained using Zeiss Axiophot microscope (Thornwood Co, USA.) were shown in FIG. 3 and FIG. 4.

According to FIGS. 3 and 4, it revealed that the mouse liver of the group 2 was filled with giant oil drops and is composed of the loosely arranged hepatocytes (FIG. 3B and FIG. 4B). But the fat accumulation in the mouse liver of the group 3, the group 4 or the group 5 was obviously decreased with comparison of group 2. Moreover, in any one of the groups 3˜5, the mouse liver was composed of the tightly arranged hepatocytes (FIG. 4C˜4E) and never was observed the giant oil drop therein (FIG. 3C˜3E).

According to the above results, it suggests that feeding with high-fat diet leads to hepatic fat accumulation. Collectively, feeding with high-fat diet on the mice would cause fatty liver and the related defects. However, treatment of the peptide of SEQ ID No.1 efficiently suppresses hepatic fat accumulation in high-fat diet fed mice. Therefore, it reveals the peptide disclosed in the present invention has therapeutic or preventive potential for fatty liver.

### Example 4

### Protein Extraction of Liver Tissue

The mouse liver of the each group was incubated in lysis buffer for tissue homogenization. The liver homogenates were placed on ice and then centrifuged at 12000 rpm for 40 minutes. After the centrifugation, the supernatants were collected and stored at −80° C. for the following examples.

### Example 5

### Western Blotting Analysis

Protein concentration of extract was determined by Lowry's protein assay method, first. The protein sample was separated in the 12% SDS polyacrylamide gel electrophoresis (hereafter referred to as SDS-PAGE) using 75 V of constant power supply. Following the SDS-PAGE, the separated proteins in the gel were transferred to PVDF membrane (GE Healthcare Life Science, Pittsburgh, Pa., USA) by using 50 V electric current for 3 hours. After proteins transfer, the PVDF membrane was incubated in 3% bovine serum albumin in TBS buffer and the primary antibodies (Santa Cruz Biotechnology, Santa Cruz, Calif., USA) were added onto the membrane for conjugation with specific protein. After the incubation with primary antibody, the horseradish peroxidase-labelled secondary antibodies were used for detection and pictures were finally taken with photographed by Fujifilm LAS-3000 (GE Healthcare Life Science).

### Example 6

### The Expression Levels of Inflammatory Cytokines

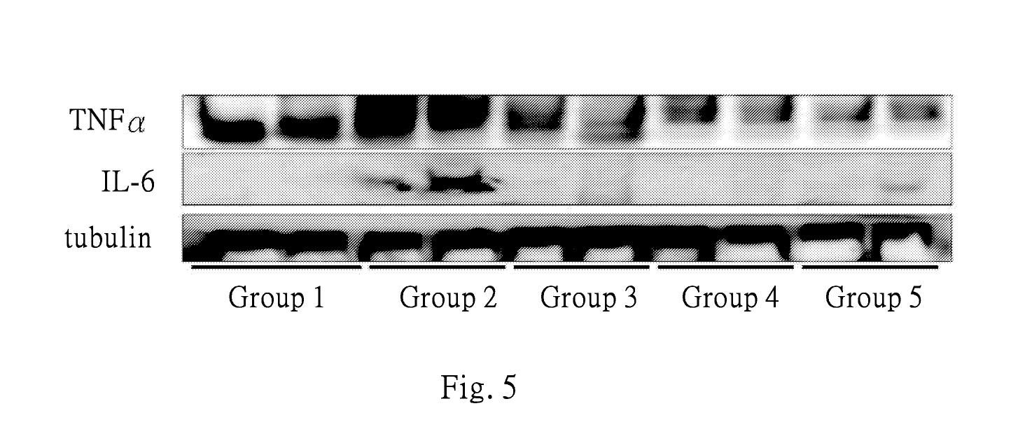

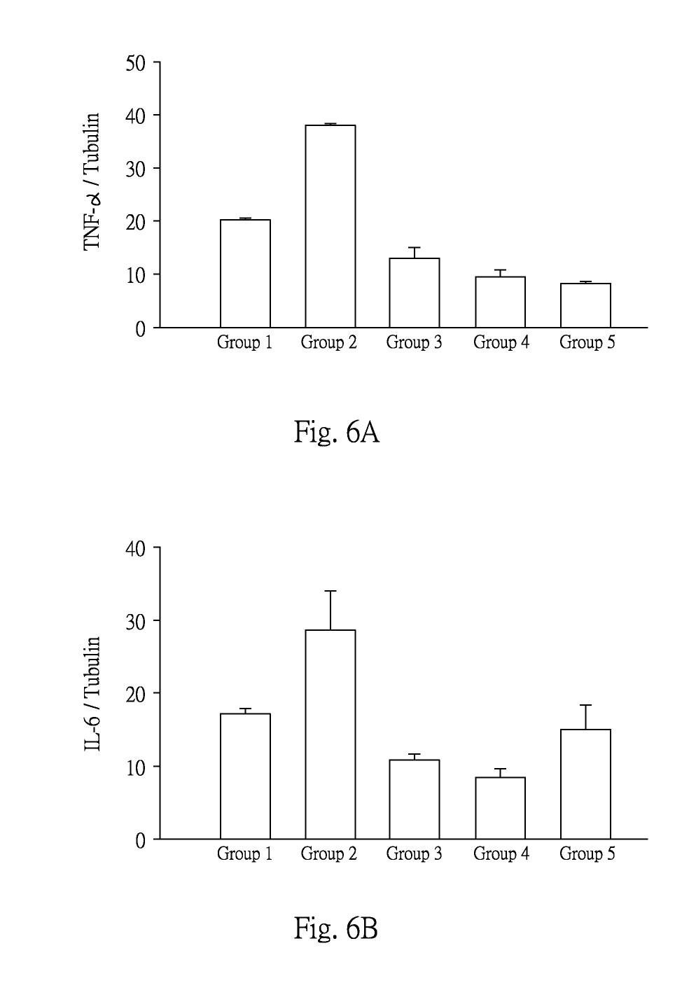

According to the methods in example 4 and 5, preparing the protein extract of mouse liver in the each group and then the expression pattern of TNFα and IL-6 in mouse liver of the each group was determined by western blotting. The results were shown in FIG. 5 and FIG. 6, wherein the expression of tubulin was utilized as internal control in FIG. 5 and FIG. 6 and the relative amount of TNFα and IL-6 in mouse liver of the each group were separately quantified and normalization with the internal control.

The bar graphs in FIG. 5 and FIG. 6 revealed that the expression levels of TNFα and IL-6 in mouse liver of the group 2 were obviously increased with comparison of group 1. Compared to the group 2, the expression levels of TNFα and IL-6 in mouse livers of the group 3, the group 4 or the group 5 were obviously reduced by administration of the peptide of SEQ ID No.1.

Collectively, the results suggest that feeding with high-fat diet will lead to elevated expression levels of TNFα and IL-6 in mouse liver. Subsequently, it activates Caspase-3 and Caspase-8 to promote liver inflammation and causes the occurrences of fatty liver and hepatic fibrosis. Inspiringly, administration of the peptide of SEQ ID No.1 on the high-fat diet fed mice will obviously reduce the expression levels of TNFα and IL-6 to inhibit the activation of Caspase-3 and Caspase-8. Therefore, the peptide disclosed in this invention is not only capable of anti-inflammation and prevention of liver damages, but also can be applied for efficient therapy or prevention of NADFL and hepatic fibrosis.

### Example 7

### The Effects of the Peptide of SEQ ID No.1 on the Expression of Apoptosis and Survival Related Proteins

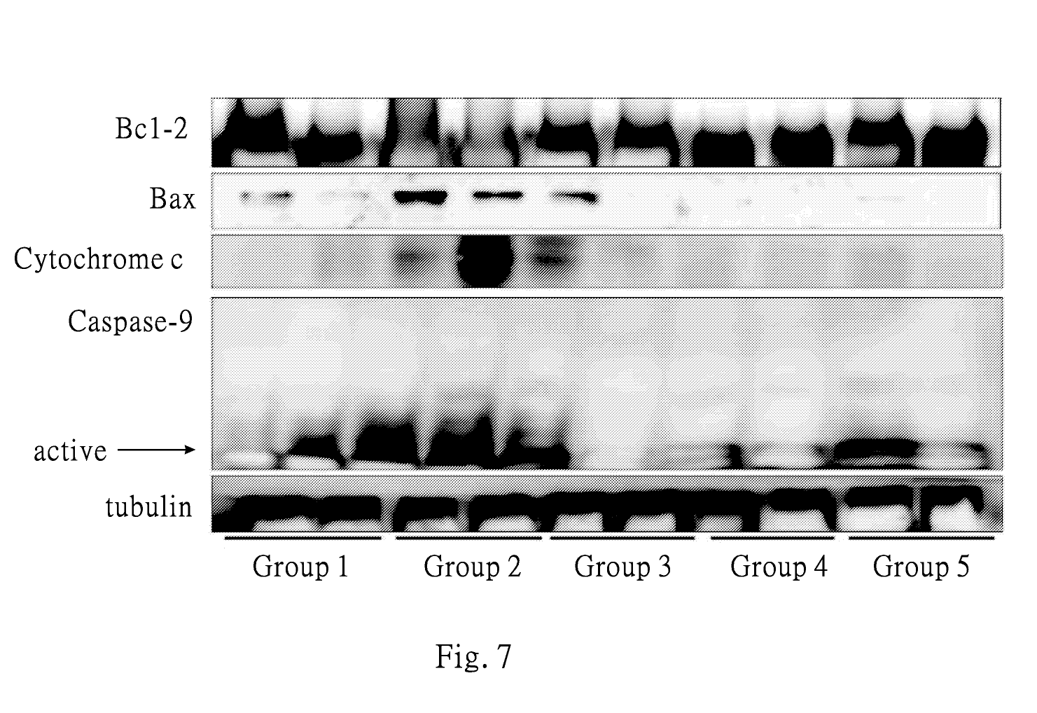

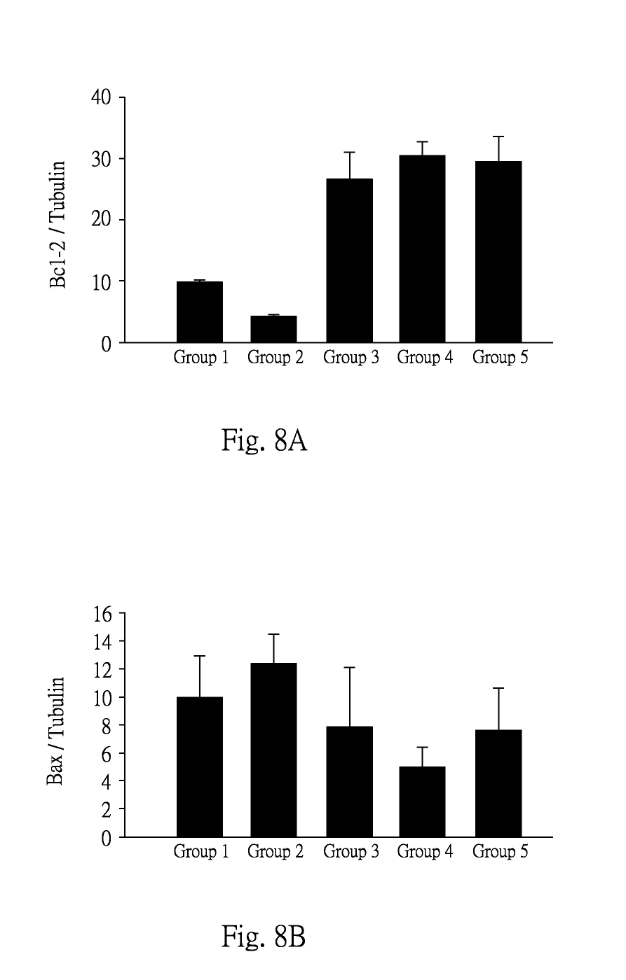

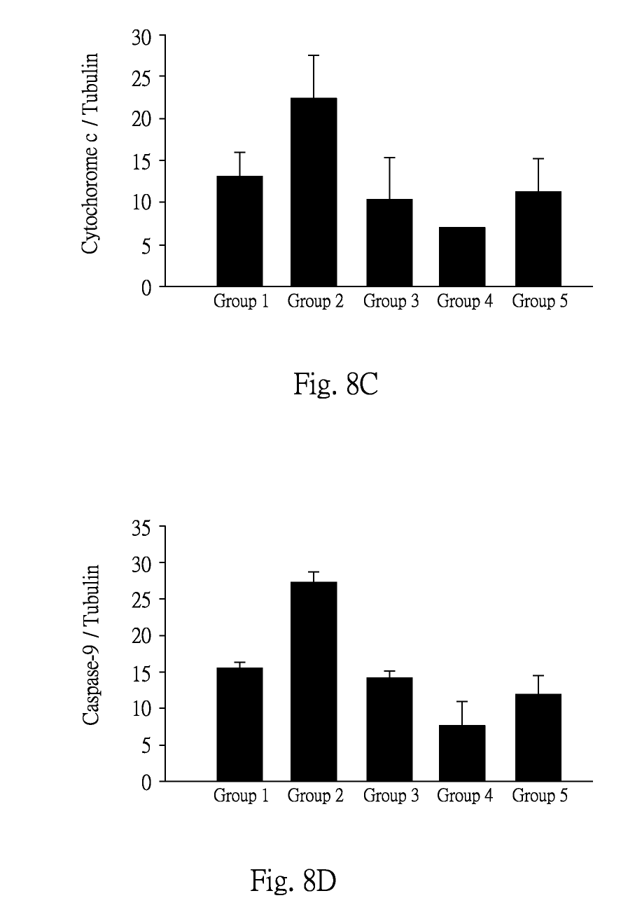

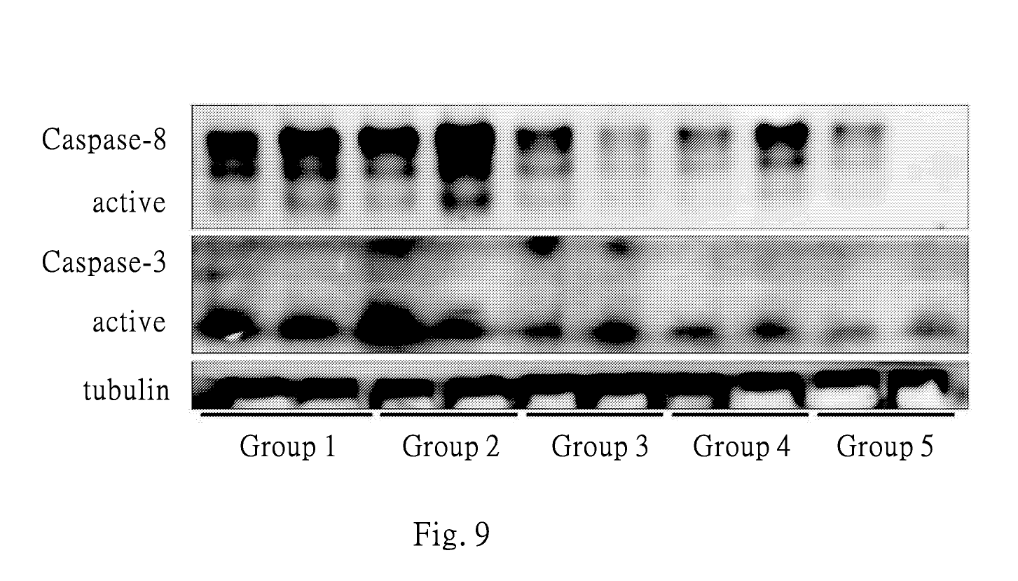

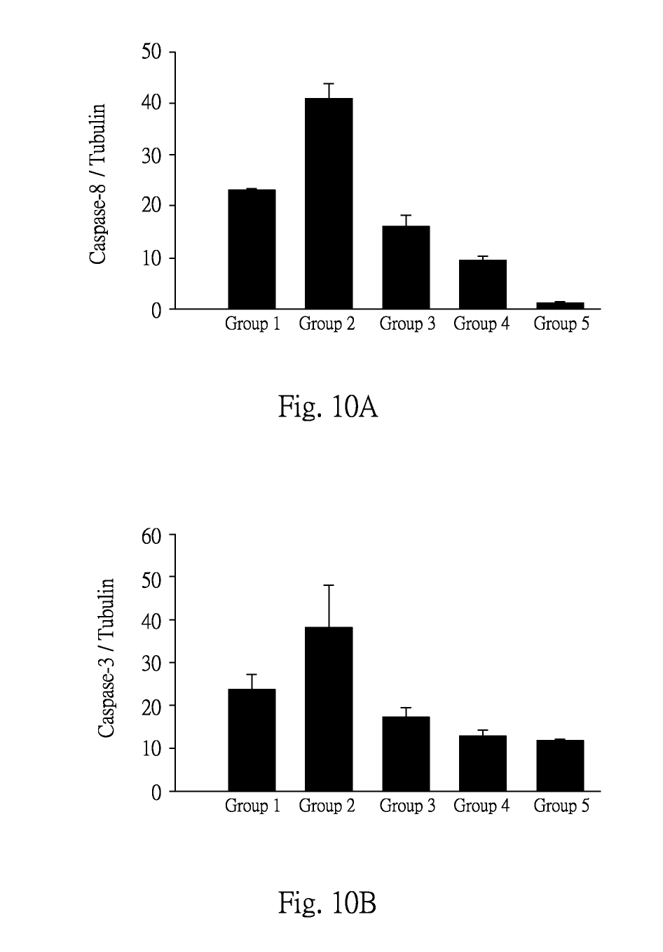

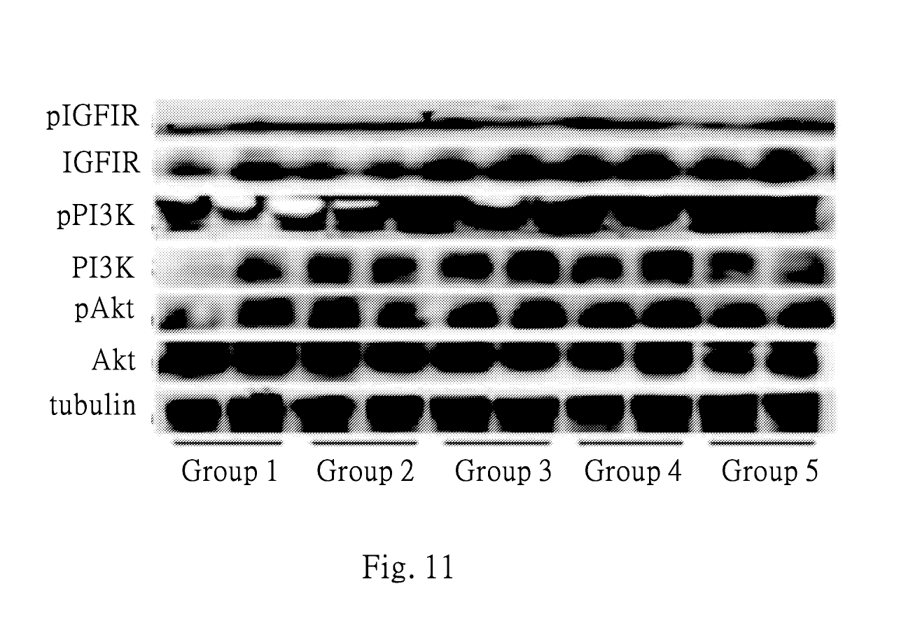

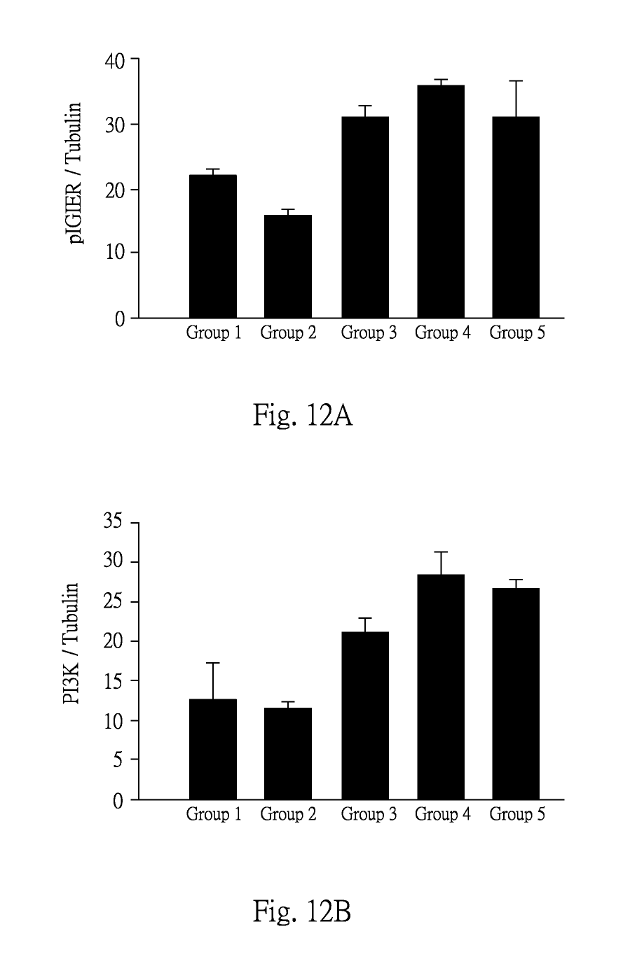

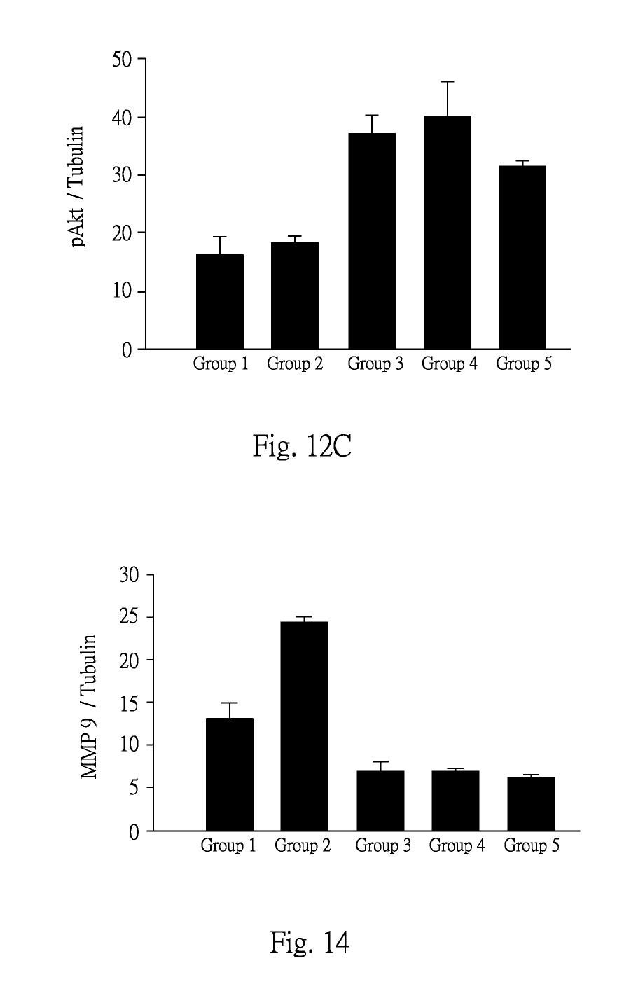

The protein extract of the mouse liver of the each group was prepared in example 4 and then to determine the expressions of apoptosis and survive related proteins using western blotting of example 5. The expression pattern of the apoptosis and survive related proteins were shown in FIG. 7 to FIG. 12, wherein the expression of tubulin was utilized as the internal control in western blot analysis in FIG. 7, FIG. 9 and FIG. 11, and the relative expression levels of the apoptosis and survive related proteins in mouse liver were normalized with tubulin for quantification in FIG. 8, FIG. 10 and FIG. 12. In addition, in FIG. 11, “pIGFIR” is the abbreviation of phosphorylated IGFIR, “pPI3K” is the abbreviation of phosphorylated PI3K and “pAKT” is the abbreviation of phosphorylated AKT.

The results in FIG. 7 to FIG. 10 were shown that the decreased expression of Bcl2 protein, but revealed the obviously increased expression of pro-apoptotic proteins including Bax, Caspase-9, Caspase-3, Caspase-8 and cytochrome c in the mouse liver of the group 2 with comparison of the group 1. Comparing with the group 2, the groups 3˜5 with administration of the peptide of SEQ ID No.1 were all elevated expression level of Bcl-2 in the mouse livers and obviously suppressed the expression levels of pro-apoptotic factors in mouse livers.

In addition, based on the results in FIG. 11 and FIG. 12, it showed that survive related proteins including IGFIR, PI3K and AKT were activated in the mouse livers of any one of the groups 3˜5, and the expression levels of the survival related proteins in the mouse livers of any one of the groups 3˜5 were obviously higher than that of the group 2.

According to FIGS. 7˜12, these results suggest that the massive hepatic fat accumulation in high-fat diet fed mice would cause liver injury and fatty liver. Furthermore, feeding with high-fat diet also further induce the expression of apoptosis-related protein to trigger hepatocyte apoptosis. However, administration of the peptide of SEQ ID No.1 on the high-fat diet fed mice will lead to activation of Bcl-2 and suppression of pro-apoptotic factors. In addition, administration of the peptide of SEQ ID No.1 also enhances the expression levels of survival factors in the high-fat diet fed mice. Therefore, even the subject with fatty liver and hepatic injury, the peptide disclosed in the present invention can inhibit hepatocyte apoptosis to treat or prevent the hepatic diseases.

### Example 8

### Effect of the Peptide of SEQ ID No.1 on Hepatic Fibrosis

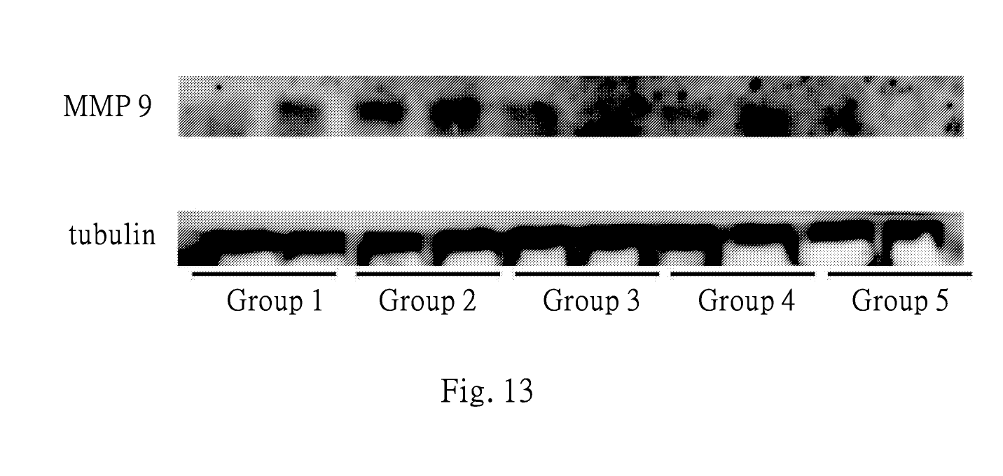

According to method in example 5, protein extract of mouse liver in the each group prepared in example 4 was subjected to determine the expression pattern and relative level of matrix metalloproteinase-9 (hereafter referred to as MMP 9). The results were shown in FIG. 13 and FIG. 14, wherein in FIG. 13, the expression of tubulin was utilized as the internal control in the western blotting and in FIG. 14, the quantified expression level of MMP-9 in the mouse liver of the each group was calculated with normalized with quantified expression level of tubulin.

According to FIGS. 13 and 14, it showed the obviously increased level of MMP-9 in mouse liver of the group 2 with comparison of the group 1. In contrast, the expression level of MMP-9 in mouse liver of any one of the groups 3˜5 was obviously suppressed with comparison of group 2.

Based on the expression of MMP-9 is utilized as the biomarker for diagnosis of hepatic fibrosis, feeding with high-fat diet results in hepatic fibrosis or elevates the risk in acquiring hepatic fibrosis according to the above results. Moreover, administration of the peptide of SEQ ID No.1 is capable of suppressing the expression level of MMP-9 in the high-fat diet fed mice. Therefore, the peptide disclosed in the present invention reveals the effects in attenuation or inhibition of hepatic fibrosis. According to these intriguing effects, the present peptide is capable of therapy or prevention of hepatic diseases.

### Example 9

### Effect of the Disclosed Peptide on Regulation of PPARα

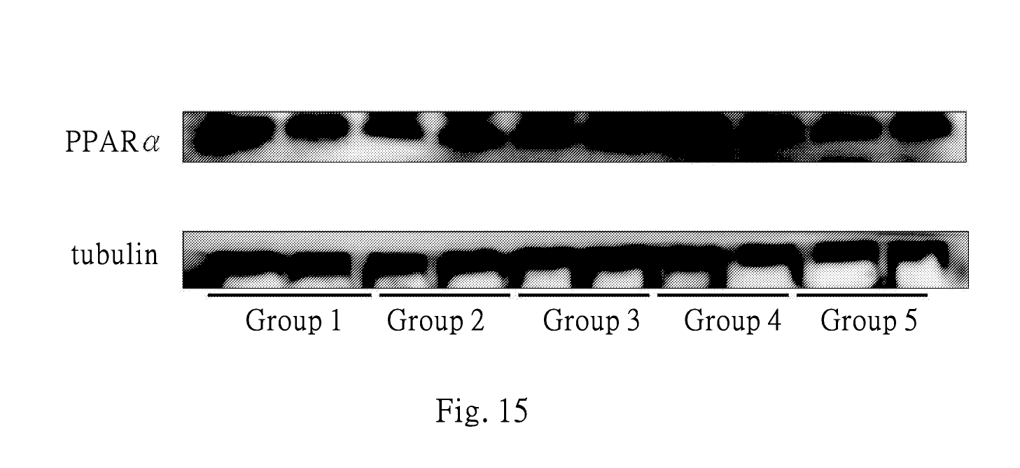

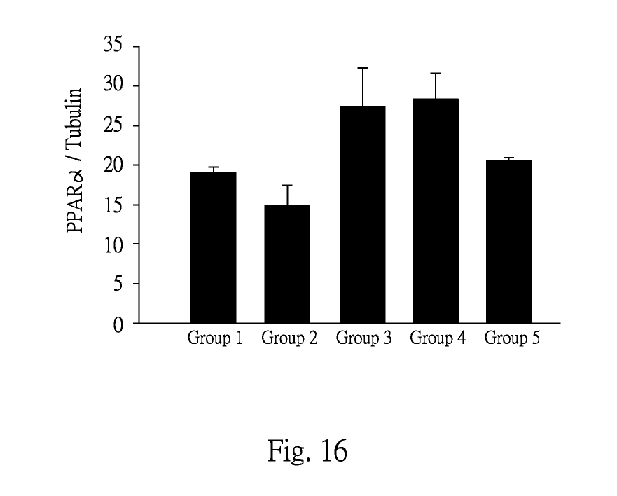

According to method in example 5, protein extract of mouse liver in each group prepared in example 4 was subjected to determine the expression pattern and relative level of PPARα. The results were shown in FIG. 15 and FIG. 16, wherein the expression of tubulin was utilized as the internal control in FIG. 15 and the quantified expression level of PPARα in the mouse livers of the each group was calculated with normalized with quantified expression level of tubulin in FIG. 16. The results in FIGS. 15 and 16 showed the obviously lower level of PPARα in mouse liver of the group 2 than that of the group 1. In contrast, the expression level of PPARα in mouse liver of any one of the groups 3˜5 was obviously increased with comparison of group 2.

Collectively, these results indicate that administration of the peptide of SEQ ID No.1 can prevent the triglyceride accumulation in liver through elevation of fatty acid metabolism rate. Therefore, the peptide disclosed in the present invention is capable of prevention or therapy for fatty liver and related diseases.

According to the examples above, the peptide containing amino acids sequence of SEQ ID No.1 actually inhibit the hepatocyte apoptosis and reduce the hepatic triglyceride accumulation. Furthermore, this peptide provides the applications for avoiding the occurrences of fatty liver, hepatic fibrosis, hepatic inflammation and other hepatic disorders. Taken together, this peptide provides the valuable functions in prevention and therapy of hepatic diseases.

It should be understood that the above-mentioned detailed description and specific examples, while indicating preferred embodiments of the invention, are given by way of illustration only. Various changes and modifications within the spirit and scope of the invention will become apparent to those skilled in the art from this detailed description. All such modifications as would be obvious to one skilled in the art are intended to be included within the scope of the following claims.

1. A method for treating or preventing liver related diseases comprises administering to a subject a composition comprising an effective amount of an isolated peptide containing an amino acid sequence of SEQ ID No.1 and at least one medical, healthy or food acceptable vehicle.

2. The method according to claim 1, wherein the liver related diseases is selected from the group consisting of hepatic fibrosis, fatty liver, hepatic inflammation, hepatic injury and apoptosis of hepatocytes.

3. The method according to claim 1, wherein the amino acid sequence of the isolated peptide is SEQ ID No.1.


SEQUENCE LISTING (1 sequence)

SEQ ID NO: 1 - protein, 4 aa, Artificial Sequence
  synthesized
VHVV